(19) United States
(12) Patent Application Publication
(10) Pub. No.: US 2005/0015038 A1
(43) Pub. Date: Jan. 20, 2005

(54) Antiseptic solution applicator

(76) Inventor: Jorge Antonio Hernandez Miramontes, Puerto Vallarta, Jalisco (MX)

Correspondence Address: SEYFARTH SHAW, 55 EAST MONROE STREET, SUITE 4200, CHICAGO, IL, 60603-5803, US

(21) Appl. No.: 10/482,423

(22) PCT Filed: Jan. 29, 2002
(86) PCT No.: PCT/MX02/00004

(30) Foreign Application Priority Data
Jul. 6, 2001 (MX) 01000018

(51) Int. Cl. A61M 35/00

(52) U.S. Cl. 604/2

(57) ABSTRACT

The invention relates to a novel sterile applicator device for antiseptic solutions. The invention eliminates the need for gloves and the packaging thereof, gases and the packaging thereof, bottles and assistance from a health care aide as well as preventing antiseptic from being wasted. The invention device combines the nine essential elements for applying an antiseptic solution in one applicator. Said applicator comprises: 1. a container; 2. the antiseptic solution; 3. a diaphragm valve; 4. a chamber cylinder; 5. sterile applicator points having variable shapes, sizes and densities; 6. a protective cover; and 7. spare disposable applicator points. The invention can be used to modify, eliminate, miniaturise, compact and combine all the elements necessary for applying an antiseptic solution in one device, thereby saving on the materials, time and human resources given over to this activity.

### BACKGROUND

In the realm of the antisepsis and disinfecting, the story of these practices and active substances began, in the modern era, with Dr. Ignaz Semmelweiss, who, by the middle of the XIX century, was able to reduce to one fourth, the death incidence of women who gave birth, by simply instructing the personnel of his hospital to disinfect the surgical niatcrial as well as their own hands before participating in the delivery process.

During the second half of that century, foundations were established for the development of some of the disciplines inherent to the modern medicine, through the disclosure of the infectious nature of many of the illness that flagellated—and still do at the present time—the population at large. We could mention for instance the discovery of the Tu berculosis Mycobacterium by Dr. Robert Koch, as the causative agent of that illness. This finding was followed by a myriad of discoveries and also by the isolation of innumerable infectious-transmissible diseases and by the research and testing of a wide variety of measures and compounds tending to controlling, preventing and treating the infectious-transmissible diseases caused by pathogenic microorganisms which include virions, virus, bacteria, fungus and protozoan.

In order to win the war against said pathogenic microorganisms, epidemiological actions were taken to detecting and isolating infectious outbreaks in the community. In the scope of the preventive medicine, precisely with the aim of preventing transmissible diseases, vaccination and prophylactic techniques were employed, including the disinfecting process by means of the application of physical or chemical methods tending to destroy and/or to control microbes on inert objects as well as the antisepsis process which unlike disinfecting, implies the destruction and/or the control of pathogenic microorganisms when found in living tissues. Said antiseptic methods may have fungicide, virucide, bactericide and protozoancide properties.

The activity of the antiseptic substances has recently been standardized by means of the phenol coefficient consisting of comparing the number of the testing antiseptic dilutions against the amount of phenol needed to destroy the microbe which is the object of the study. Taking into account the above mentioned as well as some other benefits and characteristics of the different available antiseptics, three of them have become widely accepted and used as follows: alcohol at 70%, iodine-povidone and chlorehexydine.

As mentioned before these three antiseptic substances have been widely used, especially one or them, a compound belonging to the group of the iodinephoros, known under the name of iodine-povidone consisting or iodine and polyvinylpirrolydone.

Said antiseptic substance is widely and versatily employed at the present time, and through the years it has been used as a component of different compounds. It can be found as a solution, soap, shampoo, gel, foam and aerosol. The direct or indirect application of these presentations demand special devises such as individually packed impregnated swabs, aerosol applying devices and individually packed impregnated surgical brushes. The application areas are also diverse, because said substances may be applied on the skin, on the vaginal and oral mucous membranes and also inside the organic cavities such as the thorax and peritoneum.

Summarizing, to date the antiseptic solution iodine-povidona, has been embodied in surgical brushes, in aerosol solutions, in cotton applyers, in swabs and in medical gauze. The last three items are simply impregnated in said substance and individually packed with the purpose of expediting, optimizing and saving its use. Said use is mainly aimed to achieving the antisepsis of intact skin, infected lacerations, and pre-surgical cleanings as well as the antisepsis of the oral and vaginal mucous membrane.

At the present time when antiseptic steps are taken on intact skin before performing surgery or on the treatment of skin lacerations, cellular subcutaneous tissue and muscles, generally speaking, and pursuant to the antisepsis medical protocol, said steps are performed as follows:

First it is necessary to eliminate any possible alien elements on the area to be treated.

The paramedic or medical person in charge of the treatment puts on sterile gloves on both hands, having previously extracted them from a sterile wrapping. Then an assistant would hand him/her sterile medical gauze previously extracted from sterile wrappings.

Next, the assistant opens a container (flask) and pours on the medical gauze the antiseptic fluid, most of the time in an excessive fashion. Later, the actual antiseptic treatment begins by means of the application and rubbing of the antiseptic fluid. The rubbing usually starts at the center by means of eccentric circular movements until the area to be treated becomes completely covered. That procedure is repeated for a second time. The applied antiseptic fluid generally acts from five to ten minutes and then the medical personnel proceeds with the wasting of the area and the application of sterile dressing.

If we analyze the above mentioned treatment task, we find that the following elements are necessary to perform it:
1. Sterile gloves (one pair)
2. Wrapping of the sterile gloves
3. Sterile medical gauze (a variable number of pieces, depending on the area, the depth and the contamination degree)
4. Wrapping of the sterile medical gauze
5. Container of the antiseptic substance
6. Antiseptic substance
7. Transportation carriage or medical small suitcase
8. An operator (the person in charge of the treatment)
9. An assistant (the person who assists the operator with the medical material)

It is plain to see that there are nine elements involved; seven of them related to the treatment materials and two to the personnel intervening in the performing of the antiseptic treatment of the skin or tegument.

However, all these elements (nine) to date are independent and must be gathered in order to carry out only one task: the antiseptic treatment of the skin.

Now it is possible to simplify to a minimum this process by means of the new applying device which is the subject of this patent.

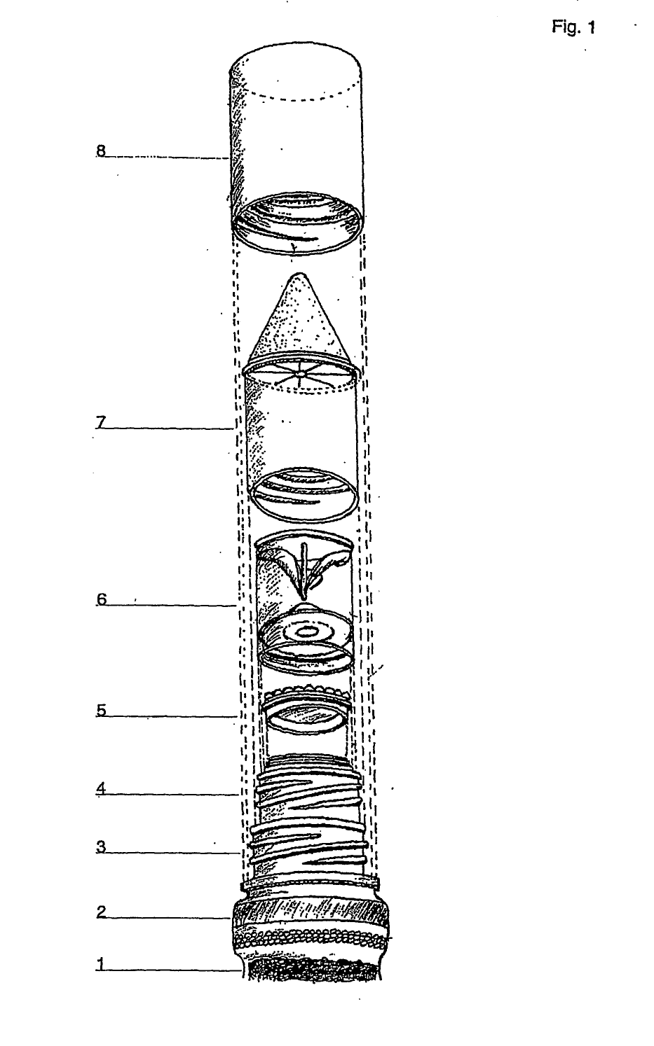

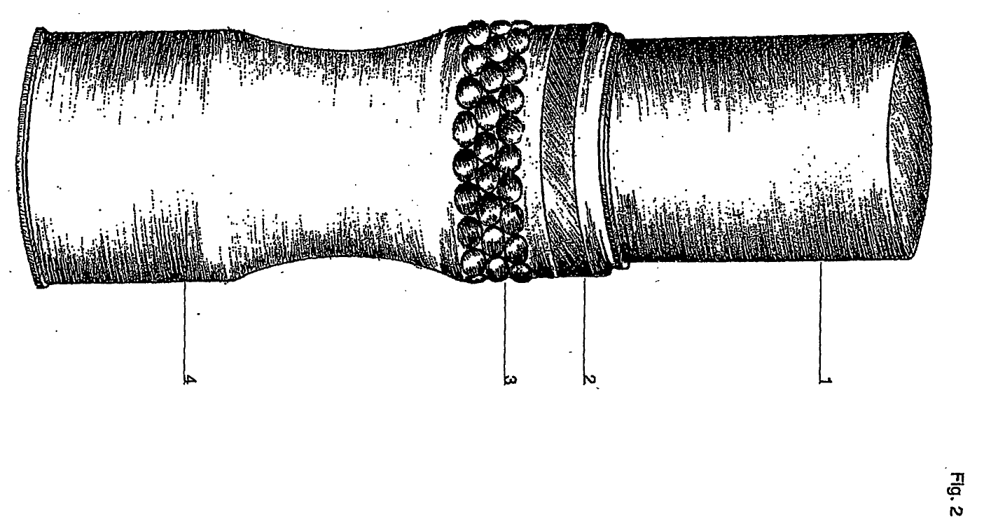

The process could be reduced to a minimum of two elements as follows:
1.—The new applying device of antiseptic solutions (FIGS. 1 and 2) and
2.—Only one person in charge of the antiseptic treatment.

It is important to point out that in several occasions the process will be reduced just to one element: the applying device of antiseptic solutions, since the patient him/herself will be able to carry out the treatment, depending on the area requiring to be cured as well as other factors such as the depth,, the extent, the time of evolution and the contamination degree of the area to be treated.

The devices with similar applications known until now are mentioned on the following lines: the U.S. Pat. No. 5,597,255 is a container with an applicator that has like a principal disadvantage the fact that it does not guarantee the sterility conditions and it does not have the possibility of disassembly the sponge, once it is contaminated. The patent U.S. Pat. No. 4,225,254 consist in a system of surgical cleaning that is complicated and is design and has the limitations to be used inside the surgical room. It has the option of recharge the antiseptic solutions using an equipment. This maneuvers require a cleaning procedure turning it unpractical. The patent FR2668371 consist of a device that has a perforated operculum, once it is used, allowing the contact of the substance with the flexible applying element; it can not be reused, due to the applying element turns contaminated at the moment of the antiseptic procedure. Under these circumstances if the antiseptic solution inside the container was not used completely, fully; that solution inside the container must be wasted, discharged, then another new device must be used turning the antiseptic procedure expensive in materials; The patent U.S. Pat. No. 474,720 describes a similar applicator, but this applicator device is designed to be used with any kind of liquids and lotions, no matter of the specific density of them; and under the concept of reusable, inasmuch as its design does not consist and is not composed by elements or including parts that insure and guarantee conditions of sterility.

The design of this new device is brought to the mind, then, with the knowledge that the similar devices knwon until now do not fulfilled with the conditions and requirements of sterility, disposable, simplicity and adaptable to be used in different types of lesions, no matter they could be deep or superficial; or on the other hand: delicate, fragil, even tender or areas of skin or death tissue that required more or less abrasive activity on it.

The new applying device of antiseptic solutions eliminates the unnecessary steps, namely by eliminating the expenses related to the sterile gloves, the wrapping of the sterile gloves, the sterile gauze, the wrapping of the sterile gauze, the transportation carriage or medical small suitcase, the excess of antiseptic solution that is pour on the wound or skin; also the medical assistant and sometimes even the operator himself, depending of the nature of the area to be treated. The use of the new applying device also reduces the size of the container flask as well as the amount of antiseptic solution to be used. It actually gathers, modifies, eliminates, reduces and compacts in a whole the elements necessary to perform the skin or teguments antisepsis without neglecting the medical protocol which should be strictly observed when carrying out the antisepsis of the tissues.

To summarize, the applying device avoids the following:
The unnecessary waste of antiseptic solution
The use of sterile gloves and the corresponding wrapping
The use of sterile gauze and the corresponding wrapping
The dependence of the medical assistant
The facilitation of the antisepsis process to such a degree that enables,
The patient to carry out him/herself the treatment procedure.

### DESCRIPTION OF THE INVENTION

The applying device of antiseptic solutions includes the following elements. Said elements are displayed in detail in the annexed drawings.

FIG. 1. It is a diagram of the elements-of the device showing their spatial relations.

FIG. 2. It is a conventional view of the exterior aspects of the device.

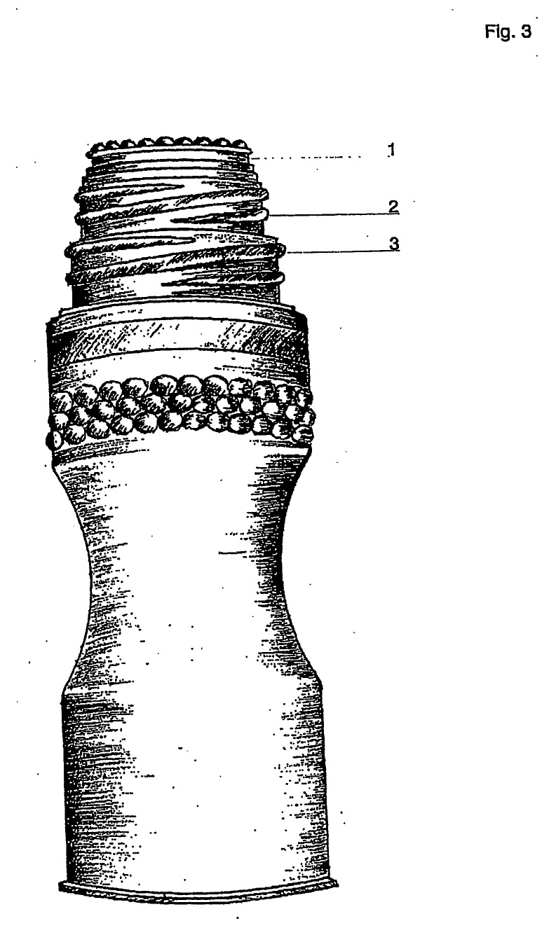

FIG. 3. It is an anterior view of the device without the protective lid and without the cylinder-chamber.

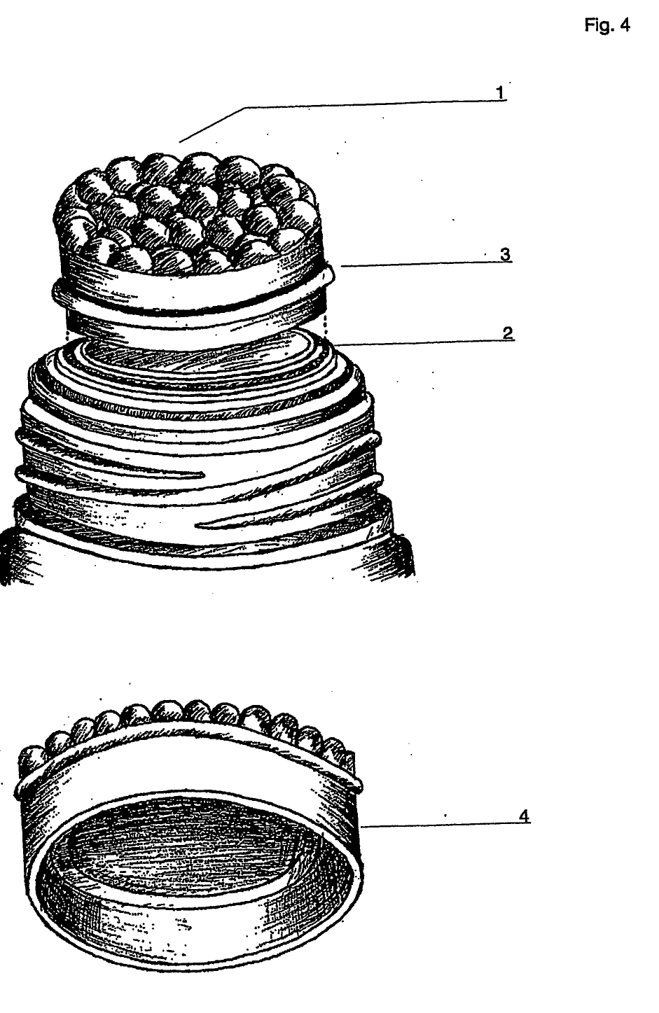

FIG. 4. It is a close view showing the details of the diaphragm and its relations.

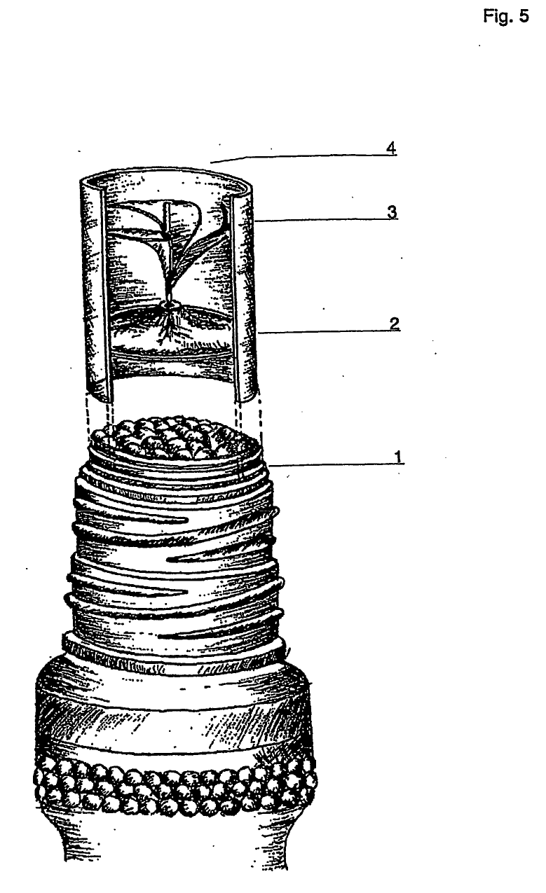

FIG. 5. It is a view of the -upper elements of the container showing the cylinder-chamber in a sagittal cutting fashion in order to appreciate its contents and its relation with the contiguos elements.

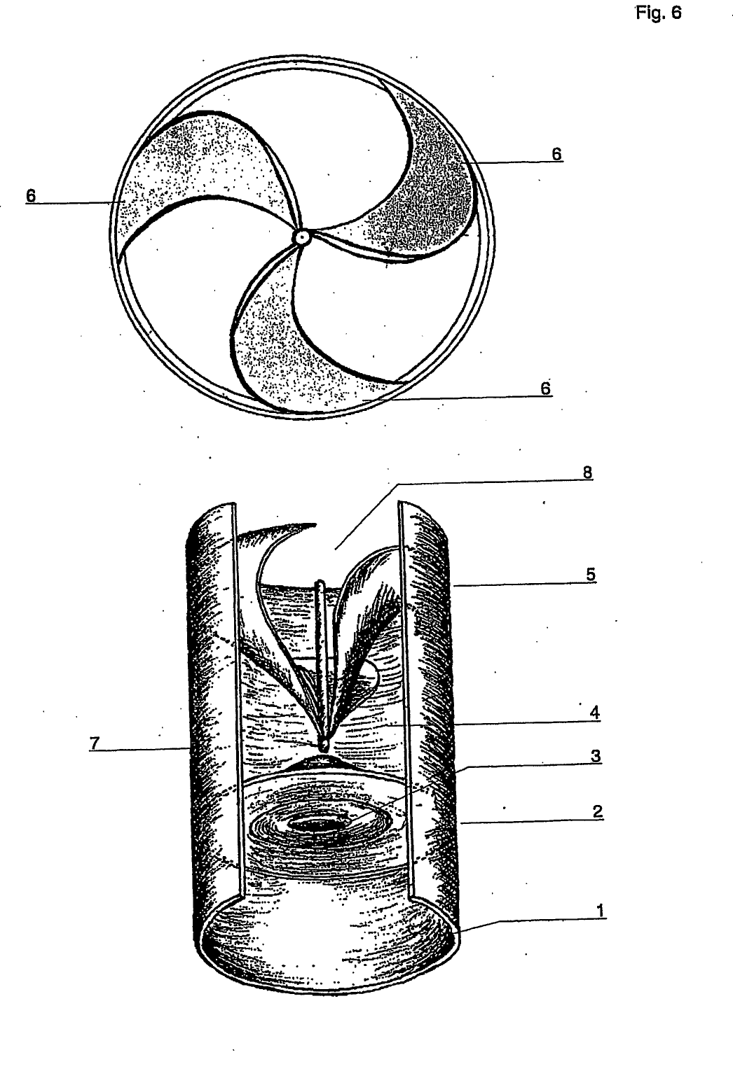

FIG. 6. The inferior section is displayed in detail by means of a sagittal cutting of the cylinder-chamber, showing its internal elements and the segments which are limited by said elements. The upper section is a crown view of the upper edge of the cylinder-chamber showing the coupling of the arched blades to the inner surface of the cylinder.

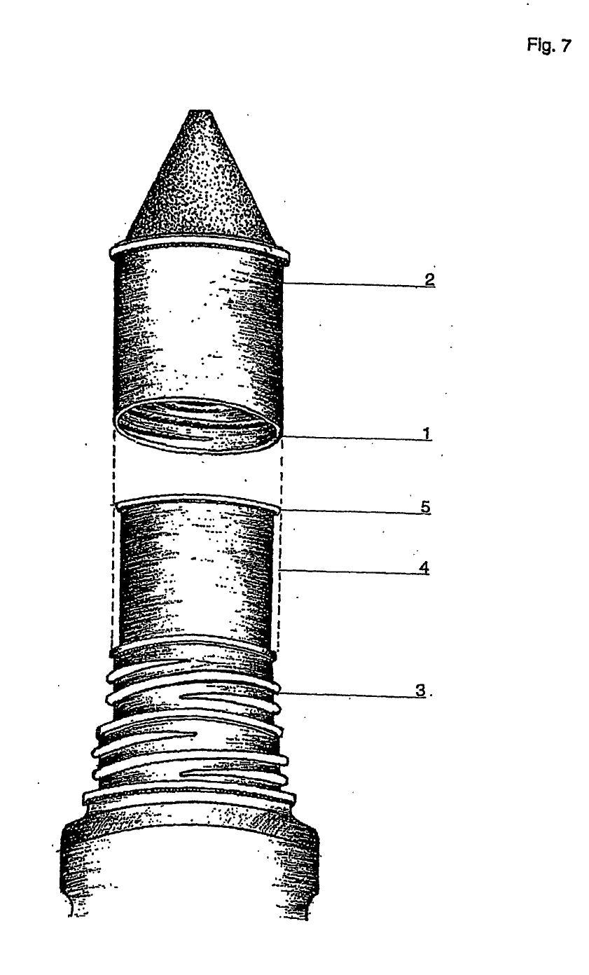

FIG. 7. It is a view of the upper section of the container showing the relation of the cylinder-chamber in situ with the sterile applying tips.

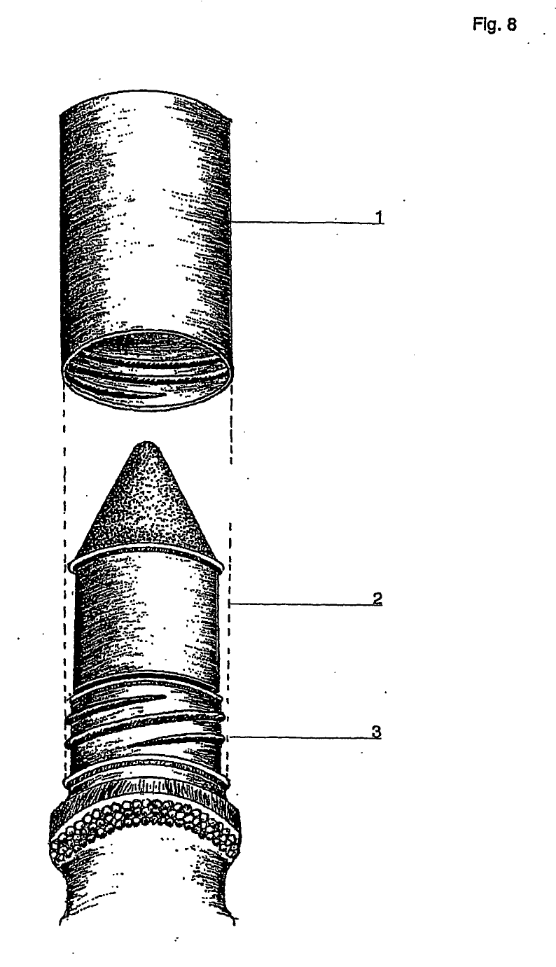

FIG. 8. It is like the previous FIG. 7, a view of the upper section of the container showing the relation and position of the applying tips.

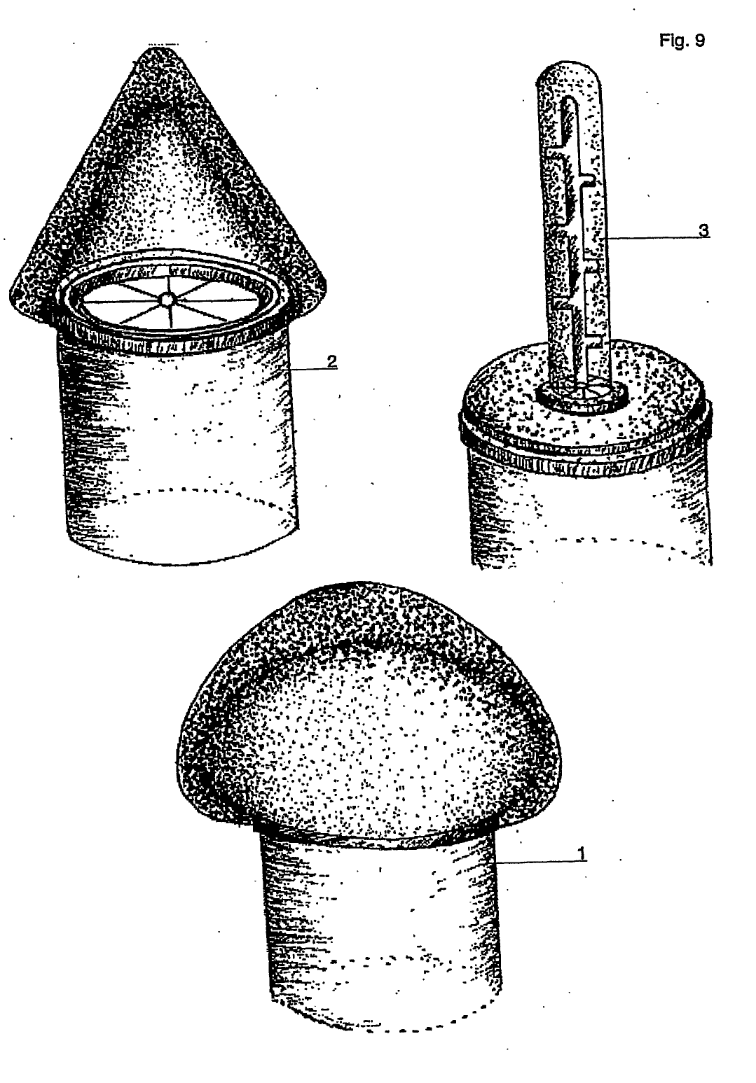

FIG. 9. In this figure are conventionally shown some of the forms and designs of the sterile applying tips.

The device is made up by a container (FIG. 2) with ergonometric characteristics and an appropriate weigh. The device may have different capacities, namely: 60, 120, 240, 500, 750, 1000 ml or any other required volume. The device is manufactured with synthetic materials. In the exterior it shows a protective lid (FIG. 2, No. 1) that covers and isolates the sterile applying tips. In the inferior third section, a concave circumference ending in an easily compressible section designed to apply pressure on it (FIG. 2 No. 3) in order to allow the antiseptic to come out from the container onto the application area. Finally there is a warning line (FIG. 2, No. 2) to point out the limit of the section that can be touched by our hands. The purpose of said line is to avoid contamination on the applying sections.

The antiseptic substances that can be used are the following: iodine-povidone, chlorhexidine gluconate (especially on iodine allergic patients) or any other present or future antiseptics.

Another component of the applying device is a diaphragm (FIG. 3 No. 1 and FIG. 4 No. 1) that leads the fluid towards a specific direction. Said diaphragm consists of a membrane of these unique materials: latex or silicon, because of the special elastic and preserve of memory-shape. Said diaphragm locks the opening of the container tube, and has been specially designed to prevent the passage of the inner fluid when the pressure is zero, but allows the liquid flow when positive pressure is exerted on the container tube. Under the area where the diaphragm is inserted the threaded area of the sterile points is located (FIG. 3, No. 2) and below said area there is another threated area (FIG. 3, No. 3) for the exterior protective lid.

If we observe closely the details of the diaphragm we can see that the surface is covered with plastic bubbles (FIG. 4, No. 1). Said bubbles are open by means of positive pressure. In the upper end of the container is a circular slot where the diaphragm fits in a hermetic fashion (FIG. 4, No. 2) in order to avoid the leaking of the fluid at this area. On the external face of the diaphragm there is a brim sealing said union ( FIG. 4, No. 3). We can see an inner section (FIG. 4, No. 4) that faces the container conduit and an external section that faces the cylinder-chamber (FIG. 4, No. 1).

The device has another element that consists of a cylinder made of a synthetic material or of a metal with the following characteristics:
To be a good receptor of the antiseptic solution
To limit the retrogressive flow of the solution
To evenly distribute the antiseptic solution

Said cylinder-chamber fits in an external edge of the slot where the diaphragm is inserted (FIG. 5, No. 1). In the inner section there are two elements that we describe as follows: a first element shaped as a concave dish (FIG. 5, No. 2 and FIG. 6, No. 2) with a central cavity (FIG. 6, No. 3) whose purpose is to allow the flowing of the antiseptic towards the next segment (FIG. 6, No. 4). When the antiseptic solution has advanced to this segment, the concave device acts as a mechanical barrier to prevent the retrogressive flow of the fluid towards the first segment of the cylinder (FIG. 6, No. 1). The second element that conforms this cylinder-chamber consists of three arched blades that are attached to the inner surface of the cylinder (FIG. 5, No. 3 and FIG. 6 No. 5), on equidistant spots (FIG. 6 No. 6 crown view of the upper section of the cylinder-chamber). Said blades couple together at the center of the cylinder in a common point forming a pole (FIG. 6 No. 7) in the direction of the concave dish. From the point the antiseptic solution would flow from the cavity to the concave dish to the next segment and immediately would fall to that common point or pole (FIG. 6 No. 7). From there on, the flow drains in a uniform fashion through the arched blades and this flowing assures that in the upper end of the cylinder (FIG. 5, No. 4 and FIG. 6 No. 8), the antiseptic will get in direct contact, bathing the whole surface of the sterile applying tip. (FIG. 7, No. 1)

The sterile applying tips may have diverse sizes and shapes especially designed to perform the antisepsis in an easy, quick, comfortable and economic fashion; made of natural textile materials or synthetic material similar to polyurethane foam.

A tipped cylinder is shown in FIG. 7 (FIG. 7, No. 2). The lower end of the internal face of the tipped cylinder is threaded (FIG. 7, No. 1). By means of this threaded section this cylindrical tip is coupled to the second threaded surface of the container (FIG. 7, No. 3). In order to achieve this coupling, the carrier cylinder with the sterile tip, slides through the external face of the cylinder-chamber (FIG. 7, No. 4). We can appreciate (FIG. 7, No. 5) an edge crowning the upper end of the cylinder-chamber.

This edge prevents that once the tipped cylinder has slid and threaded, the fluid would drip through that area. In the FIG. 8 it is possible to appreciate the relationship among the different elements and the final mounting of the sterile tipped cylinder ready to be used. Said elements are the following: (FIG. 8, No. 1) the protective lid (FIG. 8, No. 2) sterile tip and (FIG. 8, No. 3) threaded area of the container to be coupled with the protective lid.

In FIG. 9 it is possible to appreacite some sterile applying tips which are designed in different sizes, shapes and densities. The drawing shows three different tips to be used according to the circumstances: a round shaped tip (FIG. 9, No. 1) another with a conical shape (FIG. 9, No. 2) and another tip made out of metal foil or spatula (FIG. 9, No. 3).

FIG. 1

The FIG. 1 is a diagram showing all the elements of the applying device, its relationships and sequences.

Starting from the lower edge we can appreaciate the upper third of the container tube where we can see the area where positive pressure is exerted (FIG. 1, No. 1), the warning line (FIG. 1, No. 2), the threaded area of the protective tip (FIG. 1, No. 3). Next there is the threaded area of the sterile applying tip (FIG. 1, No. 4) and then the diaphragm (FIG. 1, No. 5), the cylinder-chamber (FIG. 1, No. 6), the carrier cylinder of the sterile applying tip (FIG. 1, No. 7). Finally the protective lid of the sterile tips (FIG. 1, No. 8).

The manner in which the applying device is used is simple and obvious for everybody. All you have to do is to remove the protective lid of the sterile tip in order to open it, then to exert positive pressure on the upper third, with the same hand that is holding the applying device. Next the antiseptic solution flows through the diaphragm and pours into the small cylinder-chamber. The special design of said small cylinder-chamber fulfills three purposes: it is an intermediate receptive element of the antiseptic solutions, it limits the retrogressive flow of the antiseptic and allows an even and quick distribution of the antiseptic substance on the sterile tip. The sterile tip can be made of natural textile materials such as medical gauze or synthetic materials such as polyurethane foam or others. Once the applying tip has been impregnated, the applying device can be seized with only one hand to be taken to the area to be treated. The treatment shall begin at the center and then by means of eccentric circles reach the periphery of the treatment area. The last step of this first cycle consists of the unwinding and eliminating of the sterile allready used tip, then a new one sterile tip is placed on the receptive threaded area of the device and the same operation is carried out for a second occasion. Then we wait from 5 to 8 minutes for the antiseptic solution to operate and proceed to wash and to place on the desinfected area the sterile dressing.

1. A device for applying antiseptic solutions, that comprises: a container, a diaphragm, a cylinder-chamber, one or more sterile applying tips and a protective lid. Said device is distinguished because the container has an ergonometric designed, it is sealled, it is made with flexible material with a circumference band of plastic bubbles easilly compressible. It has a threaded zone, a circle slot and a warning line on the upper end, to point out the limit of the area that must be keep out of manipulation in order to maintain the sterility conditions of the device.

2. A device for applying antiseptic solutions; pursuant to the possesion retriving No. 1, characterized because the diaphgram consists of a membrane of latex or silicon, covered of plastic bubbles of the same material. Said bubbles allow the passage of fluid in only one direction when positive pressure is applied. Said diaphragm is kept fixed in a circular slot in order to avoid any contamination of the antiseptic solutions.

3. A device to applied antiseptic solutions, pursuant to the possesion retriving No. 2, characterized because the cylinder-chamber fabricated of plastic or metal material; consists of a concave dish shaped element, with a central cavity or hole. Also consists of another element with three arched blades that are attached to the inner surface of the cylinder on equidistant points. Said blades couple together at the center of the cylinder in a common point forming a pole. Said elements limit the retrograde flow and guarantee the uniform bathing and distribution of the antiseptic solution.

4. A device for applying antiseptic solutions, pursuant to the possesion retriving No. 3, characterized because the cylinders of sterile applying tips, are made of natural textile material or synthetic material; manufactured in diverse shapes, sizes and densities to exert more or less abrasive power.

5. A device for applying antiseptic solutions, pursuant to the possesion retriving No. 4, characterized because the cylinders of sterile applying tips are pack individually, with a thread on it inner part, that allow remove the applying tip easilly in order to discard it, once they have been used on the application of the antiseptic solution, over the afected area. Said new sterile tip keep the device and the antiseptic solution perfectly sterile.

6. A device for applying antiseptic solutions, pursuant to the possesion retriving No. 5, characterized because the protective lid is threaded on its inner face, to keep the cylinder-chamber protected against any possible contamination and minimize the waste of antiseptic solution.
